(19) United States
(12) Patent Application Publication
(10) Pub. No.: US 2025/0209616 A1
(43) Pub. Date: Jun. 26, 2025

(54) AUTOMATIC SEGMENTATION OF A MEDICAL IMAGE VIA DEEP LEARNING

(71) Applicant: Novocure GmbH, Root D4 (CH)

(72) Inventors: Oren PELES ZEEVI, Haifa (IL); Amit FELDMAN, Haifa (IL)

(73) Assignee: Novocure GmbH, Root D4 (CH)

(21) Appl. No.: 18/967,867

(22) Filed: Dec. 4, 2024

Related U.S. Application Data
(60) Provisional application No. 63/614,014, filed on Dec. 22, 2023.

(51) Int. Cl. G06T 7/00; G06T 5/50; G06T 7/10; G06T 17/00; G06V 10/25; G16H 30/20; G16H 30/40

(52) U.S. Cl. CPC G06T 7/0012; G06T 5/50; G06T 7/10; G06T 17/00; G06V 10/25; G16H 30/20; G16H 30/40; G06T 2207/20084; G06T 2207/20221; G06T 2207/30096

(57) ABSTRACT

A method for reviewing a medical image including: accessing from memory a medical image of a subject, the medical image comprising voxels; generating, using a first trained machine learning model and the medical image, a first segmented medical image, the first trained machine learning model trained to generate a medical image segmenting normal tissue in a medical image; generating, using a second trained machine learning model and the medical image, a second segmented medical image, the second trained machine learning model trained to generate a medical image segmenting abnormal tissue in a medical image; combining the first segmented medical image and the second segmented medical image to obtain a segmented medical image, the segmented medical image comprising segmented normal tissue based on the first segmented medical image and segmented abnormal tissue based on the second segmented medical image.

### CROSS-REFERENCE TO RELATED APPLICATIONS

This application claims priority to U.S. Provisional Patent Application No. 63/614,014, filed Dec. 22, 2023, which is incorporated herein by reference in its entirety.

### BACKGROUND

Tumor treating fields (TTFields) are low intensity alternating electric fields within the intermediate frequency range (for example, 50 kHz to 1 MHz), which may be used to treat tumors as described in U.S. Pat. No. 7,565,205. TTFields are induced non-invasively into a region of interest by transducers placed on the patient's body and applying alternating current (AC) voltages between the transducers. Conventionally, a first pair of transducers and a second pair of transducers are placed on the subject's body. AC voltage is applied between the first pair of transducers for a first interval of time to generate an electric field with field lines generally running in the front-back direction. Then, AC voltage is applied at the same frequency between the second pair of transducers for a second interval of time to generate an electric field with field lines generally running in the right-left direction. The system then repeats this two-step sequence throughout the treatment.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A and 1B depict an example method for generating a segmented medical image and further providing recommended transducer layouts for applying TTFields to a subject based on the segmented medical image.

FIG. 2 depicts an example method for obtaining a segmented medical image.

FIG. 3 depicts an example method for obtaining a segmented medical image.

FIG. 4 depicts an example user interface to receive a user selection of a threshold for an uncertainty measure.

FIGS. 5A to 5H depict examples of a medical image processed according to an example embodiment.

FIG. 6 depicts an example system for delivering TTFields to a subject's body.

FIG. 7 depicts an example placement of transducers on a subject's head.

FIG. 8 depicts an example computer apparatus for use with the embodiments herein.

### DESCRIPTION OF EMBODIMENTS

This application describes exemplary techniques to computationally determine a segmentation of a medical image and further provide one or more recommended transducer layouts for applying TTFields to a subject based on the segmented medical image.

The inventors discovered computational techniques to vastly reduce the time needed to segment a medical image. Previously, segmentation of a medical required a user to manually segment each slice of a medical image. Since a medical image can have numerous slices and numerous voxels per slice, manual segmentation of a medical image can be a very laborious and expensive process. With the inventive techniques, a medical image of a subject may be automatically segmented based on deep learning (e.g., unsupervised trained machine learning models, deep learning trained neural networks, transformer neural networks, or convolutional neural networks (CNN)), and one or more recommended transducer layouts for applying TTFields to the subject may be generated based on the segmented medical image. The inventive techniques are particularly integrated into a practical application of providing a segmented medical image, where the segmented medical image has voxels identified as normal tissue or abnormal tissue. Further, the segmented medical image may be used in further computational processing techniques, such as generating one or more recommended transducer layouts for applying TTFields to the subject based on the segmented medical image. With the inventive techniques, the process of generating a segmented medical image may be quicker than previous manual techniques, and as such, the process of generating one or more recommended transducer layouts for applying TTFields to a subject may be quicker than prior techniques.

In some embodiments, for example, a first machine learning model may be trained to automatically segment a medical image into normal tissue and non-segmented tissue, and a second machine learning model may be trained to automatically segment a medical image into abnormal tissue and non-segmented tissue. The two segmented medical images from the two trained machine learning models may be automatically combined to obtain a segmented medical image. As such, with the inventive techniques, the process of generating a segmented medical image may be quicker than previous manual techniques and may provide for a practical application to perform automatic medical image segmentation in less computational time than prior manual techniques.

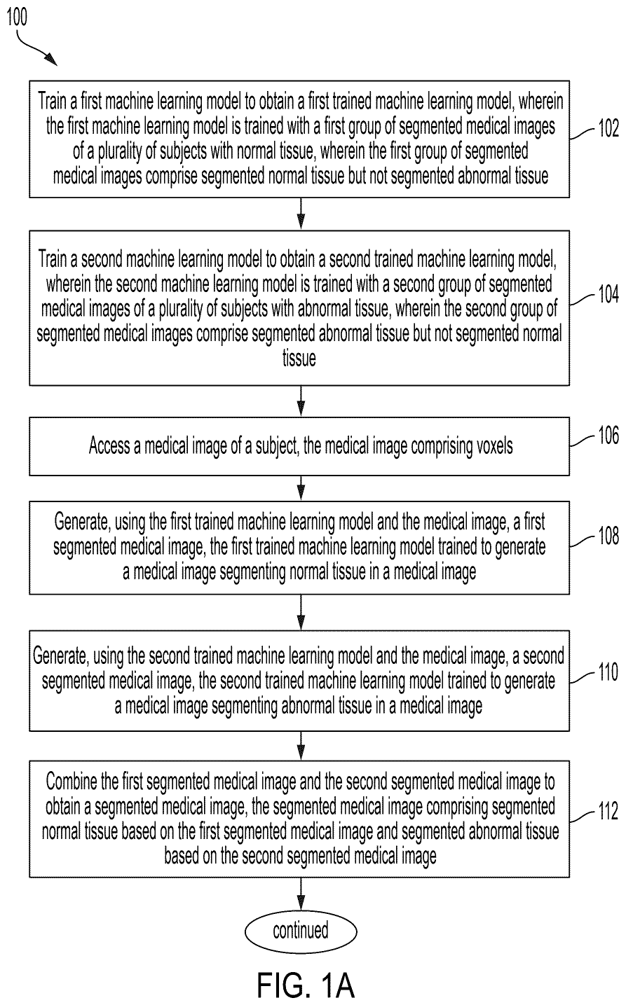

FIGS. 1A and 1B depict an example method 100 for generating a segmented medical image and further providing recommended transducer layouts for applying TTFields to a subject based on the segmented medical image. The method 100 may be implemented by a computer, the computer including one or more processors and memory accessible by the one or more processors, the memory storing instructions that when executed by the one or more processors cause the computer to perform the steps of the method 100. Modifications, additions, or omissions may be made to method 100. While an order of operations is indicated in FIGS. 1A and 1B for illustrative purposes, the timing and ordering of such operations may vary where appropriate without negating the purpose and advantages of the examples set forth in detail herein.

The method 100 includes automatic segmentation of a medical image. In some embodiments, the medical image may include one of a magnetic resonance imaging (MRI) medical image, a computed tomography (CT) medical image, or a positron emission tomography (PET) medical image. A medical image may include a plurality of voxels and a plurality of slices, where each slice has a plurality of voxels.

The method 100 may include, at step 102, training a first machine learning model to obtain a first trained machine learning model, where the first machine learning model is trained with a first group of segmented medical images of a plurality of subjects with normal tissue. In some embodiments, the first group of segmented medical images may include segmented normal tissue but not segmented abnormal tissue. As one example, the first machine learning model is trained to generate a medical image segmenting normal tissue in a medical image and is not trained to generate a medical image segmenting abnormal tissue in a medical image.

At step 104, the method 100 may include training a second machine learning model to obtain a second trained machine learning model, where the second machine learning model may be trained with a second group of segmented medical images of a plurality of subjects with abnormal tissue. In some embodiments, the second group of segmented medical images may include segmented abnormal tissue but not segmented normal tissue. As one example, the second trained machine learning model may be trained to generate a medical image segmenting abnormal tissue in a medical image and is not trained to generate a medical image segmenting normal tissue in a medical image.

In some embodiments, the normal tissue mentioned herein may include at least one organ, and the abnormal tissue mentioned herein may include at least one of a tumor, an improperly functioning organ, or a resection area. As one example, the normal tissue may include at least one of a lung or a heart, and the abnormal tissue may include at least one of a tumor in a chest of a subject, a collapsed lung, or a fluid-filled lung. As another example, the normal tissue may include at least one of a brain, and the abnormal tissue may include at least one of a tumor in a head of a subject or a resection area in a head of a subject.

The first trained machine learning model and the second trained machine may include one or more algorithms and/or structures. In some embodiments, the first trained machine learning model and the second trained machine learning model may each be a deep learning trained neural network. In some embodiments, the first trained machine learning model or the second trained machine learning model may each be an unsupervised trained machine learning model. In some embodiments, the first trained machine learning model and the second trained machine learning model may each include at least one of a projective adversarial network (PAN), a variational autoencoder (VAE), or an unsupervised trained neural network. In some embodiments, the first trained machine learning model and the second trained machine learning model may utilize transformer neural network or CNN. As one example, the first trained machine learning model and the second trained machine learning model may each be a transformer model utilizing the transformer neural network. As another example, the first trained machine learning model and the second trained machine learning model may utilize the CNN architecture, for example, a U-Net architecture. In some embodiments, the first trained machine learning model and the second trained machine learning model may each be based on the same machine learning model but may also be based on different machine learning models.

At step 106, the method 100 may include accessing a medical image of a subject, where the medical image includes voxels. In some embodiments, the medical image may be stored at and accessible from a computer memory locally or over a network.

At step 108, the method 100 may include generating, using the first trained machine learning model and the medical image from step 106, a first segmented medical image, where the first trained machine learning model is trained to generate a medical image segmenting normal tissue in a medical image, where the first segmented medical image may indicate segmented normal tissue. In some embodiments, the first segmented medical image may include voxels having segmentation label data. In some embodiments, the segmentation label data of the first segmented medical image may include one of a segmented normal tissue label or a non-segmented tissue label. In some embodiments, the segmentation label data of the first segmented medical image may include a label uncertainty measure for each label.

At step 110, the method 100 may include generating, using the second trained machine learning model and the medical image from step 106, a second segmented medical image, where the second trained machine learning model is trained to generate a medial image segmenting abnormal tissue in a medical image, where the second segmented medical image may indicate segmented abnormal tissue. In some embodiments, the second segmented medical image may include voxels having segmentation label data. In some embodiments, the segmentation label data of the second segmented medical image may include one of a segmented abnormal tissue label or a non-segmented tissue label. In some embodiments, the segmentation label data of the second segmented medical image may include a label uncertainty measure for each label.

The segmentation label data of the first segmented medical image and the second segmented medical image may include non-segmented tissue label. In some embodiments, instead of including non-segmented tissue label, the first segmented medical image and the second segmented medical image may include no label data for voxels that were not segmented as normal tissue or as abnormal tissue.

At step 112, the method 100 may include combining the first segmented medical image and the second segmented medical image to obtain a segmented medical image, where the segmented medical image may include the segmented normal tissue based on the first segmented medical image from step 108 and the segmented abnormal tissue based on the second segmented medical image from step 110. In some embodiments, the combination may include prioritizing the second segmented medical image over the first segmented medical image. In some embodiments, the combination may include prioritizing the first segmented medical image over the second segmented medical image.

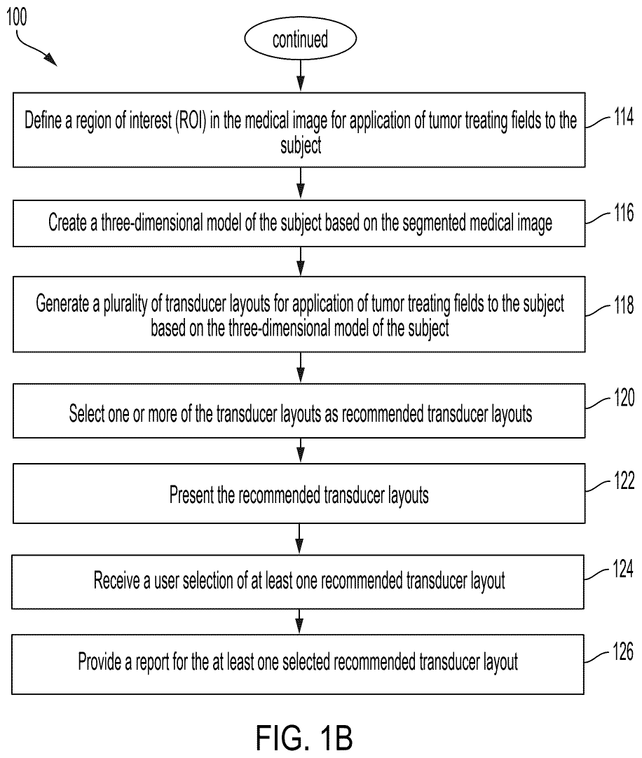

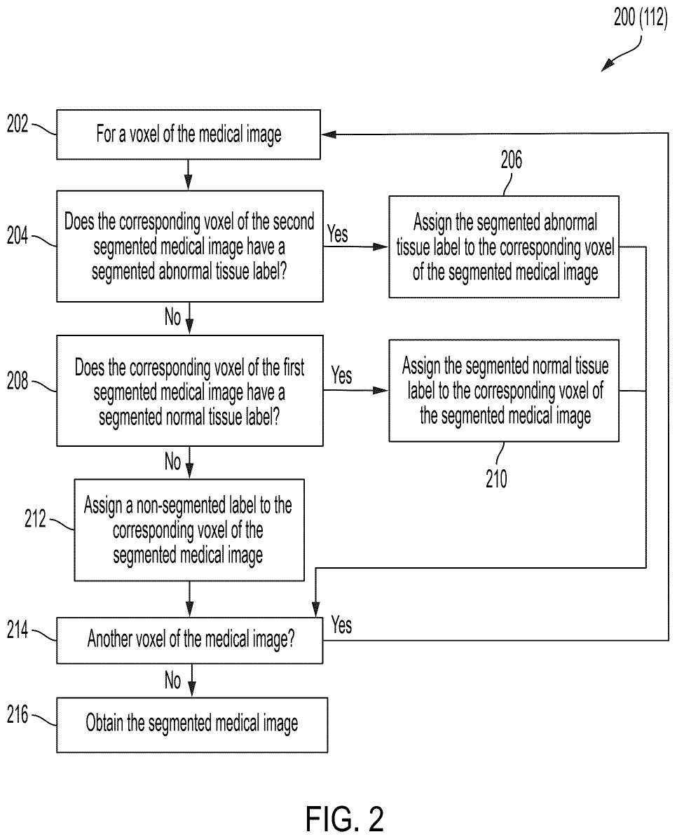

In some embodiments, step 112 may be performed by assigning segmentation label data for each voxel of the medical image based on segmentation label data of a corresponding voxel of either the first segmented medical image or the second segmented medical image. In some embodiments, step 112 may be performed by assigning segmentation label data for each voxel of the medical image based on an uncertainty measure of segmentation label data of a corresponding voxel of either the first segmented medical image or the second segmented medical image. In some embodiments, each voxel may be assigned segmentation label data based on prioritizing the first segmented medical image over the second segmented medical image, or vice versa. In some embodiments, a voxel may be assigned a segmented normal tissue label, a segmented abnormal tissue label, or a non-segmented tissue label. In some embodiments, a voxel may be assigned a segmented normal tissue label or a segmented abnormal tissue label, and if a voxel is assigned a non-segmented tissue label, the voxel is reassigned a segmented normal tissue label. More details of step 112 are discussed further below with respect to FIG. 2 and FIG. 3.

At step 114, the method 100 may include defining a region of interest (ROI) in the segmented medical image for application of TTFields to the subject. The ROI may define where the TTFields are to focus. As one example, the ROI may include all of the abnormal tissue. As one example, the ROI may include at least part of the abnormal tissue. In some embodiments, the ROI may be defined based on user input or may be defined partially or entirely by the computer.

At step 116, the method 100 may include creating a three-dimensional (3D) model of the subject based on the segmented medical image from step 112, where the 3D model of the subject may include the ROI. In some embodiments, the 3D model may include a 3D conductivity map depicting electrical conductivity of tissues of the subject. In some embodiments, the 3D model may be generated by the computer and may be based on user input.

In some embodiments, creating the 3D model may include performing calculations to determine conductivity of the tissues of the subject based on the segmented medical image and the tissue types therein. As one example, creating the 3D model may include assigning tissue types and associated conductivities to voxels of the 3D model of the subject. In some embodiments, the 3D model may be created based on user input, such as user approval on a 3D conductivity map associated with the 3D model.

At step 118, the method 100 may include generating a plurality of transducer layouts for application of TTFields to the subject based on the 3D model of the subject. The transducer layouts may define one or more locations, relative to the subject, for placing one or more transducers. In some embodiments, a transducer layout may include four locations on the subject to place four respective transducers, such as on a head or torso of the subject. In some embodiments, the plurality of the transducer layouts may include two locations on the subject to place two respective transducers, such as on a head or torso of the subject. In some embodiments, the transducer may include one electrode element or a plurality of electrode elements. The electrode element may be any suitable type or material. For example, at least one electrode element may include a ceramic dielectric layer, a polymer film, and/or the like including combinations and/or multiples thereof.

At step 120, the method 100 may include selecting one or more of the transducer layouts as one or more recommended transducer layouts. In some embodiments, one, two, three, four or more transducer layouts may be selected as recommended transducer layouts. In some embodiments, the selection may be based on user input. For example, the user input may indicate which one or more transducer layouts are selected as the one or more recommend transducer layouts.

At step 122, the method 100 may include presenting the recommended transducer layouts on a display. In some embodiments, the recommended transducer layouts may be presented on a display through one or more output devices for a user's consideration.

At step 124, the method 100 may include receiving a user selection of at least one recommended transducer layout. The user selection may indicate a preferred transducer layout to deliver the TTFields to the ROI of the subject.

At step 126, the method 100 may include providing a report for the at least one selected recommended transducer layout, where the report indicates the selection result from step 124.

Turning to FIG. 2, FIG. 2 illustrates an example method 200 to perform step 112 in FIG. 1A to obtain the segmented medical image. The method 200 may be implemented by a computer. Modifications, additions, or omissions may be made to the example illustrated in FIG. 2. While an order of operations is indicated in FIG. 2 for illustrative purposes, the timing and ordering of such operations may vary where appropriate without negating the purpose and advantages of the examples set forth in detail herein.

At step 202, the method 200 may include selecting a voxel in the medical image. Later steps 204 to 212 are repetitively executed for voxels selected in the medical image to obtain the segmented medical image.

At step 204, the method 200 may include determining, for the voxel selected at step 202, if a corresponding voxel of the second segmented medical image has a segmented abnormal tissue label. Upon determining yes, the process may go to step 206. Otherwise, the process may go to step 208.

At step 206, the method 200 may include assigning the segmented abnormal tissue label to the corresponding voxel of the segmented medical image. Specifically, based on the determination at step 204, if a corresponding voxel of the second segmented medical image has a segmented abnormal tissue label, the voxel of the segmented medical image may be assigned the segmented abnormal tissue label of the corresponding voxel of the second segmented medical image. The process may go to step 214 afterwards.

At step 208, the method 200 may include determining, for the voxel selected at step 202, if a corresponding voxel of the first segmented medical image has a segmented normal tissue label. Upon determining yes, the process may go to step 210. Otherwise, the process may go to step 212.

At step 210, the method 200 may include assigning the segmented normal tissue label to the voxel of the segmented medical image. Specifically, based on the determinations at steps 204 and 208, if a corresponding voxel of the second segmented medical image does not have a segmented abnormal tissue label, and if a corresponding voxel of the first segmented medical image has a segmented normal tissue label, the voxel of the segmented medical image may be assigned the segmented normal tissue label of the corresponding voxel of the first segmented medical image. The process may go to step 214 afterwards.

At step 212, the method 200 may include assigning a non-segmented label to the voxel of the segmented medical image. Specifically, based on the determinations at steps 204 and 208, if a corresponding voxel of the second segmented medical image does not have a segmented abnormal tissue label, and if a corresponding voxel of the first segmented medical image does not have a segmented normal tissue label, the voxel of the segmented medical image may be assigned the non-segmented tissue label in the first segmented medical image or the non-segmented tissue label in the second segmented medical image. In some embodiments, instead of assigning a non-segmented tissue label to the voxel of the segmented medical image in step 212, the voxel of the segmented medical image may not be assigned any segmentation tissue label. The process may go to step 214 afterwards.

At step 214, the method 200 may include determining if there is another voxel of the medical image that has not been selected at step 202. Upon determining yes, the process may go back to step 202, and repeat steps 202 to 212 for the another voxel. Upon determining no, the process may go to step 216.

At step 216, the method 200 may conclude with obtaining the segmented medical image, where the voxels of the segmented medical image are labeled based on steps 202 to 214.

Turning to FIG. 3, FIG. 3 illustrates an example method 300 to perform step 112 in FIG. 1A to obtain the segmented medical image. The method 300 may be implemented by a computer. Modifications, additions, or omissions may be made to the example illustrated in FIG. 3. While an order of operations is indicated in FIG. 3 for illustrative purposes, the timing and ordering of such operations may vary where appropriate without negating the purpose and advantages of the examples set forth in detail herein.

At step 302, the method 300 may include selecting a voxel in the medical image. Later steps 304 to 316 are repetitively executed for voxels selected in the medical image to obtain the segmented medical image.

At step 304, the method 300 may include determining, for the voxel selected at step 302, if a corresponding voxel of the second segmented medical image has an uncertainty measure greater than a too-uncertain threshold, and if a corresponding voxel of the first segmented medical image has an uncertainty measure greater than the too-uncertain threshold. Upon determining yes to both determinations, the process may go to step 306. Otherwise, the process may go to step 308.

At step 306, the method 300 may include assigning a non-segmented label to the corresponding voxel of the segmented medical image. Specifically, upon determining at step 304 that the corresponding voxel of the second segmented medical image has an uncertainty measure greater than the too-uncertain threshold, and that the corresponding voxel of the first segmented medical image has an uncertainty measure greater than the too-uncertain threshold, assign the non-segmented tissue label to the voxel of the segmented medical image. In some embodiments, instead of assigning a non-segmented tissue label to the voxel of the segmented medical image in step 306, the voxel of the segmented medical image may not be assigned any segmentation tissue label. The process may go to step 318 afterwards.

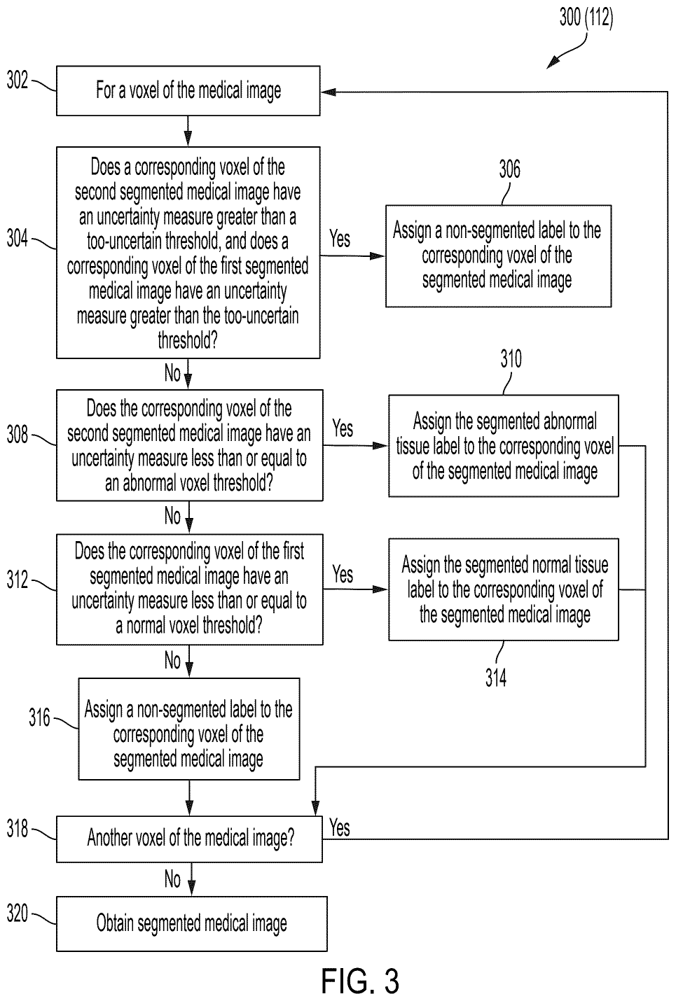

At step 308, the method 300 may include determining, for the voxel selected at step 302, if the corresponding voxel of the second segmented medical image has an uncertainty measure less than or equal to an abnormal voxel threshold. In some embodiments, the abnormal voxel threshold may be based on a user selection and may be, for example, a user-selected abnormal voxel threshold. In some embodiments, the method 300 may include providing a user interface to receive a user selection of the abnormal voxel threshold. FIG. 4 depicts an example user interface to receive a user selection for such a threshold. Upon determining yes, the process may go to step 310. Upon determining no, the step may go to step 312.

At step 310, the method 300 may include assigning the segmented abnormal tissue label to the corresponding voxel of the segmented medical image. In some embodiments, step 310 may include assigning the segmented abnormal tissue label to the voxel of the segmented medical image based on the user-selected abnormal voxel threshold. The process may go to step 318 afterwards.

At step 312, the method 300 may include determining, for the voxel selected at step 302, if the corresponding voxel of the first segmented medical image has an uncertainty measure less than or equal to a normal voxel threshold. In some embodiments, the normal voxel threshold may be a received user selection and may be, for example, a user-selected normal voxel threshold. In some embodiments, the method 300 may include providing a user interface to receive a user selection of the normal voxel threshold. FIG. 4 depicts an example user interface to receive a user selection for such a threshold. Upon determining yes, the process may go to step 314; upon determining no, the process may go to step 316.

At step 314, the method 300 may include assigning the segmented normal tissue label to the corresponding voxel of the segmented medical image. In some embodiments, step 314 may include assigning the segmented normal tissue label to the voxel of the segmented medical image based on the user-selected normal voxel threshold. The process may go to step 318 afterwards.

At step 316, the method 300 may include assigning a non-segmented label to the corresponding voxel of the segmented medical image upon that all determinations at steps 304, 308, and 312 are no. The process may go to step 318 afterwards.

At step 318, the method 300 may include determining if there is another voxel of the medical image that has not been selected. Upon determining yes, the process may return to step 302 to pick another voxel of the medical image and repeat steps 304 to 316 for the another voxel of the medical image. In some embodiments, instead of assigning a non-segmented tissue label to the voxel of the segmented medical image in step 318, the voxel of the segmented medical image may not be assigned any segmentation tissue label. Upon determining no, the process may go to step 320.

At step 320, the method 300 may conclude with obtaining the segmented medical image, where the voxels of the segmented medical image are labeled based on steps 302 to 318.

In FIG. 3, the threshold comparisons in steps 304, 308, and 310 are based on an uncertainty measure being low corresponding to a high certainty. If instead, an uncertainty measure being low corresponds to a low certainty, the threshold comparisons in steps 304, 308, and 310 are reversed (e.g., in step 304, flow goes to step 306 if the uncertainty measure is less than a too-uncertain threshold and to step 308 otherwise).

FIG. 4 depicts an example user interface 400 to receive a user selection of a threshold for an uncertainty measure. In some embodiments, a user interface, such as the user interface 400 of FIG. 4, may be used to receive a user selection of the too-uncertain threshold, the abnormal voxel threshold, and the normal voxel threshold discussed above in regards to FIG. 3. The user interface 400 may include a slider 402 that may be selected and moved by the user between a minimum 404 (e.g., 0%) and a maximum 406 (e.g., 100%). The value for the threshold may be a percentage or a number on a scale. Instead of a slider, other user interfaces may be used, such as, for example, a dial, selectable buttons, or a field for entry of a number.

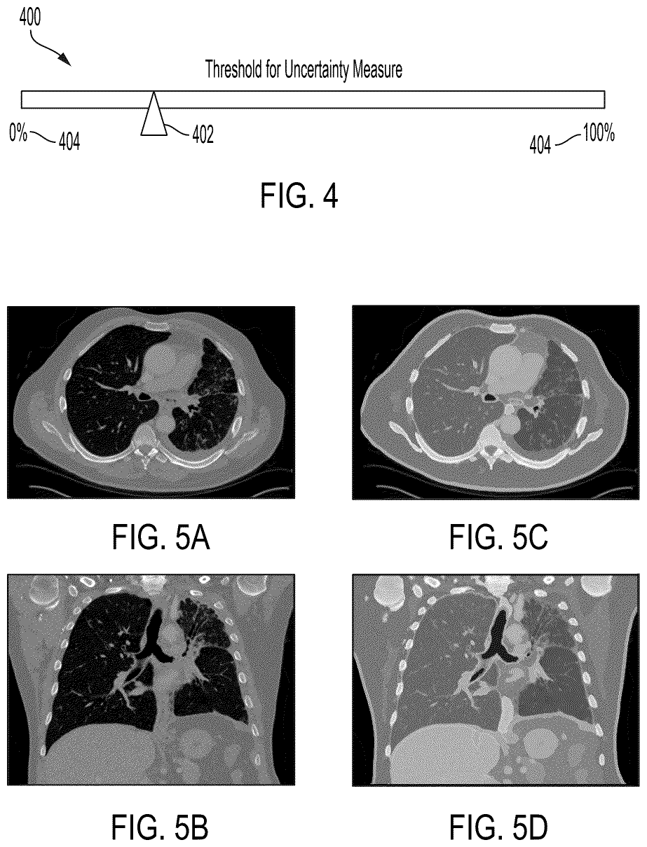

FIGS. 5A to 5H depict examples of a medical image of a subject processed according to an example embodiment. FIGS. 5A, 5C, 5E, and 5G depict a transverse plane view of a torso of a subject, and FIGS. 5B, 5D, 5F, and 5H depict a coronal plane view of the torso of the subject. FIGS. 5A and 5B depict the medical image prior to automatic segmenting according to an example embodiment and accessed at step 106. FIGS. 5C and 5D depict the medical image with segmentation of normal tissue generated at step 108. FIGS. 5E and 5F depict the medical image with segmentation of abnormal tissue generated at step 110. FIGS. 5G and 5H depict the segmented medical image generated at step 112, which includes the combination of the segmentation of normal tissue from step 108 and the segmentation of abnormal tissue from step 110.

### Exemplary Apparatuses

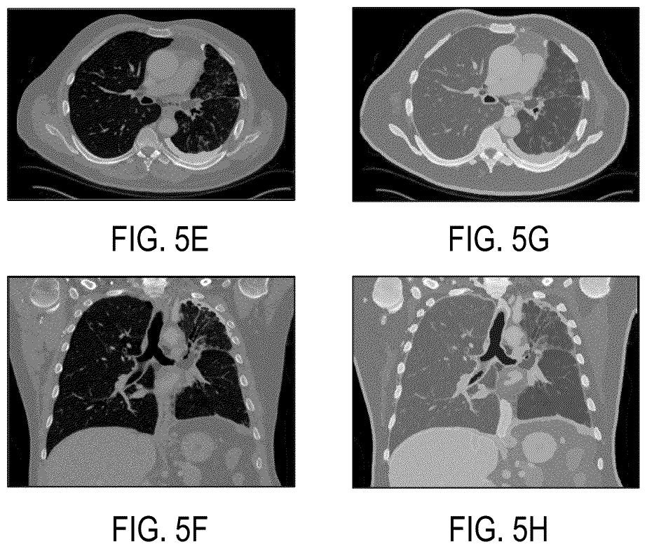

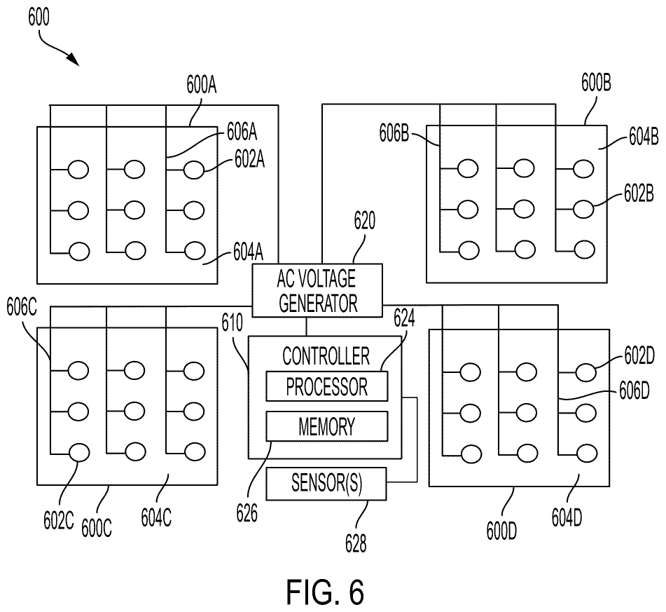

FIG. 6 depicts an example apparatus 600 to apply alternating electric fields (e.g., TTFields) to a subject's body. The system may be used for treating a target region of a subject's body with an alternating electric field (e.g., TTFields). As an example, the target region may be in the subject's brain, and an alternating electric field may be delivered to the subject's body via two pairs of transducers positioned on a head of the subject's body (such as, for example, in FIG. 7, which has four transducers 700). As an example, the target region may be in the subject's torso, and an alternating electric field may be delivered to the subject's body via two pairs of transducers positioned on at least one of a thorax, an abdomen, or one or both thighs of the subject's body. As an example, a single pair of transducers may be used. Other transducer placements on the subject's body may be possible.

The example apparatus 600 depicts an example having four transducers (or “transducer arrays”) 600A-D. Each transducer 600A-D may include substantially flat electrode elements 602A-D positioned on a substrate 604A-D and electrically and physically connected (e.g., through conductive wiring 606A-D). The substrates 604A-D may include, for example, cloth, foam, flexible plastic, and/or conductive medical gel. Two transducers (e.g., 600A and 600D) may be a first pair of transducers configured to apply an alternating electric field to a target region of the subject's body. The other two transducers (e.g., 600B and 600C) may be a second pair of transducers configured to similarly apply an alternating electric field to the target region.

The transducers 600A-D may be coupled to an AC voltage generator 620, and the system may further include a controller 610 communicatively coupled to the AC voltage generator 620. The controller 610 may include a computer having one or more processors 624 and memory 626 accessible by the one or more processors. The memory 626 may store instructions that when executed by the one or more processors control the AC voltage generator 620 to induce alternating electric fields between pairs of the transducers 600A-D according to one or more voltage waveforms and/or cause the computer to perform one or more methods disclosed herein. The controller 610 may monitor operations performed by the AC voltage generator 620 (e.g., via the processor(s) 624). One or more sensor(s) 628 may be coupled to the controller 610 for providing measurement values or other information to the controller 610.

In some embodiments, the voltage generation components may supply the transducers 600A-D with an electrical signal having an alternating current waveform at frequencies in a range from about 50 kHz to about 1 MHz and appropriate to deliver TTFields treatment to the subject's body

The electrode elements 602A-D may be capacitively coupled. As an example, the electrode elements 602A-D may be ceramic electrode elements coupled to each other via conductive wiring 606A-D. When viewed in a direction perpendicular to its face, the ceramic electrode elements may be circular shaped or non-circular shaped. In other embodiments, the array of electrode elements may not be capacitively coupled, and there is no dielectric material (such as ceramic, or high dielectric polymer layer) associated with the electrode elements.

The structure of the transducers 600A-D may take many forms. The transducers may be affixed to the subject's body or attached to or incorporated in clothing covering the subject's body. The transducer may include suitable materials for attaching the transducer to the subject's body. For example, the suitable materials may include cloth, foam, flexible plastic, and/or a conductive medical gel. The transducer may be conductive or non-conductive.

The transducer may include any desired number of electrode elements (e.g., one electrode element, or more than one electrode element). Various shapes, sizes, and materials may be used for the electrode elements. Any constructions for implementing the transducer (or electric field generating device) for use with embodiments of the invention may be used as long as they are capable of (a) delivering TTFields to the subject's body and (b) being positioned at the locations specified herein. In some embodiments, at least one electrode element of the first, the second, the third, or the fourth transducer may include at least one ceramic disk that is adapted to generate an alternating electric field. In some embodiments, at least one electrode element of the first, the second, the third, or the fourth transducer includes a polymer film that is adapted to generate an alternating field.

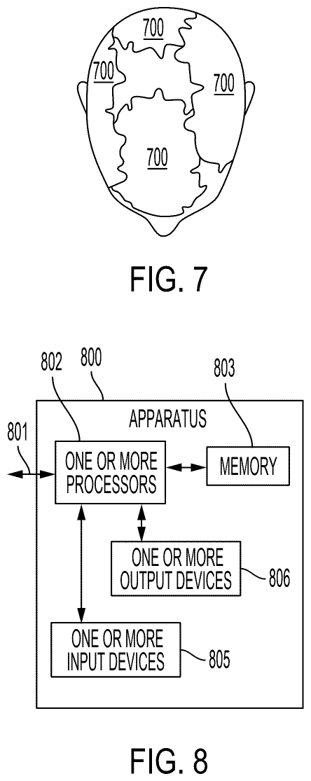

FIG. 8 depicts an example computer apparatus for use with one or more embodiments described herein. As an example, the apparatus 800 may be a computer to implement certain inventive techniques disclosed herein, such as a computing device to implement a method for reviewing a medical image and/or such as a computing device to implement the method for generating a segmented medical image and further providing recommended transducer layouts for applying TTFields to a subject based on the segmented medical image in FIGS. 1 and 2. As an example, some or all of the steps in the method illustrated in FIGS. 1 and 2 may be performed on a single apparatus 800. As an example, a computing device used for generating the segmented medical image may be implemented by a first apparatus 800, and a computing device used for providing recommended transducer layouts for applying TTFields to a subject based on the segmented medical image may be implemented by a second apparatus 800. As an example, the apparatus 800 may be used as the controller 610 of FIG. 6, or as a separate computer apparatus located remote from the controller 610.

The apparatus 800 may include one or more processors 802, memory 803, one or more input devices 805, and one or more output devices 806.

Input to the apparatus 800 may be provided by one or more input devices 805, provided from one or more input devices in communication with the apparatus 800 via link 801 (e.g., a wired link or a wireless link; e.g., with a direct connection or over a network), and/or provided from another computer(s) in communication with the apparatus 800 via link 801. As an example, based on input 801, the one or more processors 802 may generate control signals to control the AC voltage generator 620. As an example, the input 801 may be user input. As an example, the input 801 may be from another computer in communication with the apparatus 800.

Output for the apparatus 800 may be provided by one or more output devices 806, provided to one or more output devices in communication with the apparatus 800 via link 801, and/or provided from another computer(s) in communication with the apparatus 800 via link 801. The one or more output devices 806 may provide the status of the operation of the invention, such as generated transducer layouts, recommended transducer layouts, and other operational information. The output device(s) 806 may provide visualization data according to certain embodiments of the invention.

In some embodiments, one or more input devices 805 and one or more output devices 806 may be combined into one or more unitary input/output devices (e.g., a touch screen).

In some embodiments, based on input from one or more input devices 805 or input from outside the apparatus 800 via the link 801, the one or more processors 802 may perform operations as described herein. As an example, user input may be received from the one or more input devices 805. As an example, input may be from another computer in communication with the apparatus 800 via link 801. As an example, input may be from one or more input devices in communication with the apparatus 800 via link 801.

In some embodiments, the one or more processors 802 may perform operations as described herein and provide results of the operations as output. As an example, output may be provided to the one or more output devices 806. As an example, output may be provided to another computer in communication with the apparatus 800 via link 801. As an example, output may be provided to one or more output devices in communication with the apparatus 800 via link 801.

The memory 803 may be accessible by the one or more processors 802 so that the one or more processors 802 may read information from and write information to the memory 803. The memory 803 may store instructions that, when executed by the one or more processors 802, implement one or more embodiments described herein. The memory 803 may be a non-transitory computer readable medium (or a non-transitory processor readable medium) containing a set of instructions thereon for reviewing a medical image and/or for selecting one or more transducer layouts for delivering tumor treating fields to a subject, wherein when executed by a processor (such as one or more processors 802), the instructions cause the processor to perform one or more methods discussed herein.

The apparatus 800 may be an apparatus for reviewing a medical image and/or for selecting one or more transducer layouts for delivering tumor treating fields to a subject, the apparatus including: one or more processors (such as one or more processors 802); and memory (such as memory 803) accessible by the one or more processors, the memory storing instructions that when executed by the one or more processors, cause the apparatus to perform one or more methods described herein.

The memory 803 may be a non-transitory processor readable medium containing a set of instructions thereon for reviewing a medical image and/or for selecting one or more transducer layouts for delivering tumor treating fields to a subject, wherein when executed by one or more processors (such as one or more processors 802), the instructions cause the one or more processors to perform one or more methods described herein.

### ILLUSTRATIVE EMBODIMENTS

The invention includes other illustrative embodiments (“Embodiments”) as follows.
Embodiment 1: A computer-implemented method for reviewing a medical image, the method comprising: accessing from memory a medical image of a subject, the medical image comprising voxels; generating, using a first trained machine learning model and the medical image, a first segmented medical image, the first trained machine learning model trained to generate a medical image segmenting normal tissue in a medical image; generating, using a second trained machine learning model and the medical image, a second segmented medical image, the second trained machine learning model trained to generate a medical image segmenting abnormal tissue in a medical image; combining the first segmented medical image and the second segmented medical image to obtain a segmented medical image, the segmented medical image comprising segmented normal tissue based on the first segmented medical image and segmented abnormal tissue based on the second segmented medical image.
Embodiment 2: The method of Embodiment 1, wherein the first segmented medical image comprises voxels having segmentation label data, the segmentation label data comprising one of a segmented normal tissue label or a non-segmented tissue label, and wherein the second segmented medical image comprises voxels having segmentation label data, the segmentation label data comprising one of a segmented abnormal tissue label or a non-segmented tissue label.
Embodiment 3: The method of Embodiment 2, wherein the segmentation label data of the first segmented medical image further comprises a label uncertainty measure, and wherein the segmentation label data of the second segmented medical image further comprises a label uncertainty measure.
Embodiment 4: The method of Embodiment 1, wherein combining the first segmented medical image and the second segmented medical image to obtain the segmented medical image comprises prioritizing the second segmented medical image over the first segmented medical image.
Embodiment 4A: The method of Embodiment 1, wherein combining the first segmented medical image and the second segmented medical image to obtain the segmented medical image comprises prioritizing the first segmented medical image over the second segmented medical image.
Embodiment 5: The method of Embodiment 1, wherein combining the first segmented medical image and the second segmented medical image to obtain the segmented medical image comprises: for a voxel of the medical image, assigning segmentation label data for the voxel of the medical image based on segmentation label data of a corresponding voxel of either the first segmented medical image or the second segmented medical image.
Embodiment 6: The method of Embodiment 1, wherein combining the first segmented medical image and the second segmented medical image to obtain the segmented medical image comprises: for a voxel of the segmented medical image, if a corresponding voxel of the second segmented medical image has a segmented abnormal tissue label, assigning the segmented abnormal tissue label to the voxel of the segmented medical image, and if the corresponding voxel of the second segmented medical image does not have the segmented abnormal tissue label, and if a corresponding voxel of the first segmented medical image has a segmented normal tissue label, assigning the segmented normal tissue label to the voxel of the segmented medical image.
Embodiment 7: The method of Embodiment 1, wherein for a voxel of the segmented medical image: if a corresponding voxel of the second segmented medical image has a segmented abnormal tissue label, the voxel of the segmented medical image has the segmented abnormal tissue label of the corresponding voxel of the second segmented medical image, and if the corresponding voxel of the second segmented medical image does not have the segmented abnormal tissue label, and if a corresponding voxel of the first segmented medical image has a segmented normal tissue label, the voxel of the segmented medical image has the segmented normal tissue label of the corresponding voxel of the first segmented medical image.
Embodiment 8: The method of Embodiment 7, wherein for a voxel of the segmented medical image: if the corresponding voxel of the second segmented medical image does not have a segmented abnormal tissue label, and if the corresponding voxel of the first segmented medical image does not have a segmented normal tissue label, the voxel of the segmented medical image has a non-segmented tissue label.
Embodiment 9: The method of Embodiment 1, wherein combining the first segmented medical image and the second segmented medical image to obtain the segmented medical image comprises: for a voxel of the medical image, assigning segmentation label data for the voxel of the medical image based on an uncertainty measure of segmentation label data of the corresponding voxel of either the first segmented medical image or the second segmented medical image.
Embodiment 10: The method of Embodiment 1, wherein combining the first segmented medical image and the second segmented medical image to obtain the segmented medical image comprises: for a voxel of the segmented medical image, if a corresponding voxel of the second segmented medical image has an uncertainty measure less than or equal to an abnormal voxel threshold, assigning a segmented abnormal tissue label to the voxel of the segmented medical image, and if the corresponding voxel of the second segmented medical image has an uncertainty measure greater than an abnormal voxel threshold, and if a corresponding voxel of the first segmented medical image has an uncertainty measure less than or equal to a normal voxel threshold, assigning the segmented normal tissue label to the voxel of the segmented medical image.
Embodiment 11: The method of Embodiment 10, wherein combining the first segmented medical image and the second segmented medical image to obtain the segmented medical image further comprises: for a voxel of the segmented medical image, if a corresponding voxel of the second segmented medical image has an uncertainty measure greater than an abnormal voxel threshold, and if a corresponding voxel of the first segmented medical image has an uncertainty measure greater than a normal voxel threshold, assigning a non-segmented tissue label to the voxel of the segmented medical image.
Embodiment 12: The method of Embodiment 10, wherein combining the first segmented medical image and the second segmented medical image to obtain the segmented medical image further comprises: for a voxel of the segmented medical image, if a corresponding voxel of the second segmented medical image has an uncertainty measure greater than a too-uncertain threshold, and if a corresponding voxel of the first segmented medical image has an uncertainty measure greater than a too-uncertain threshold, assigning a non-segmented tissue label to the voxel of the segmented medical image.
Embodiment 13: The method of Embodiment 10, wherein the first segmented medical image comprises voxels having segmentation label data, the segmentation label data comprising one of a segmented normal tissue label or a non-segmented tissue label, the segmentation label data further comprising a label uncertainty measure for each label, wherein the second segmented medical image comprises voxels having segmentation label data, the segmentation label data comprising one of a segmented abnormal tissue label or a non-segmented tissue label, the segmentation label data further comprising a label uncertainty measure for each label.
Embodiment 14: The method of Embodiment 10, wherein combining the first segmented medical image and the second segmented medical image to obtain the segmented medical image further comprises: receiving a user selection of the abnormal voxel threshold; assigning the segmented abnormal tissue label to the voxel of the segmented medical image based on the user-selected abnormal voxel threshold; receiving a user selection of the normal voxel threshold; and assigning the segmented normal tissue label to the voxel of the segmented medical image based on the user-selected normal voxel threshold.
Embodiment 14A: The method of Embodiment 10, wherein combining the first segmented medical image and the second segmented medical image to obtain the segmented medical image further comprises: providing a user interface to receive a user selection of the abnormal voxel threshold; and providing a user interface to receive a user selection of the normal voxel threshold.
Embodiment 15: The method of Embodiment 1, wherein the first trained machine learning model comprises a transformer neural network or a convolutional neural network, wherein the second trained machine learning model comprises a transformer neural network or a convolutional neural network.
Embodiment 15A: The method of Embodiment 1, wherein the first trained machine learning model is a deep learning trained neural network.
Embodiment 15B: The method of Embodiment 1, wherein the first trained machine learning model is an unsupervised trained machine learning model.
Embodiment 15C: The method of Embodiment 1, wherein the first trained machine learning model comprises at least one of a projective adversarial network (PAN), a variational autoencoder (VAE), or an unsupervised trained neural network.
Embodiment 15D: The method of Embodiment 1, further comprising: training a machine learning model to obtain the first trained machine learning model, wherein the machine learning model is trained with segmented medical images of subjects with normal tissue, wherein the segmented medical images comprise segmented normal tissue, wherein the segmented medical images do not comprise segmented abnormal tissue; and training a machine learning model to obtain the second trained machine learning model, wherein the machine learning model is trained with segmented medical images of subjects with abnormal tissue, wherein the segmented medical images comprise segmented abnormal tissue, wherein the segmented medical images do not comprise segmented normal tissue.
Embodiment 16: The method of Embodiment 1, wherein the first trained machine learning model is trained to generate a medical image segmenting normal tissue in a medical image and is not trained to generate a medical image segmenting abnormal tissue in a medical image, wherein the second trained machine learning model is trained to generate a medical image segmenting abnormal tissue in a medical image and is not trained to generate a medical image segmenting normal tissue in a medical image.
Embodiment 17: The method of Embodiment 1, wherein the normal tissue comprises at least one organ, and the abnormal tissue comprises at least one of a tumor, an improperly functioning organ, or a resection area.
Embodiment 17A: The method of Embodiment 1, wherein the normal tissue comprises at least one of a lung or a heart, and the abnormal tissue comprises at least one of a tumor in a chest of a subject, a collapsed lung, or a fluid-filled lung.
Embodiment 17B: The method of Embodiment 1, wherein the normal tissue comprises at least one of a brain, and the abnormal tissue comprises at least one of a tumor in a head of a subject or a resection area in a head of a subject.
Embodiment 17C: The method of Embodiment 1, wherein the medical image comprises one of a magnetic resonance imaging (MRI) medical image, a computed tomography (CT) medical image, or a positron emission tomography (PET) medical image.
Embodiment 18: The method of Embodiment 1, further comprising: defining a region of interest (ROI) in the medical image for application of tumor treating fields to the subject; creating a three-dimensional model of the subject based on the segmented medical image, the three-dimensional model of the subject including the region of interest; generating a plurality of transducer layouts for application of tumor treating fields to the subject based on the three-dimensional model of the subject; selecting at least two of the transducer layouts as recommended transducer layouts; presenting the recommended transducer layouts; receiving a user selection of at least one recommended transducer layout; and providing a report for the at least one selected recommended transducer layout.
Embodiment 18A: A non-transitory processor readable medium containing a set of instructions thereon for reviewing a medical image, wherein when executed by a processor, the instructions cause the processor to perform a method comprising: accessing from memory a medical image of a subject, the medical image comprising voxels; generating, using a first trained machine learning model and the medical image, a first segmented medical image, the first trained machine learning model trained to generate a medical image segmenting normal tissue in a medical image; generating, using a second trained machine learning model and the medical image, a second segmented medical image, the second trained machine learning model trained to generate a medical image segmenting abnormal tissue in a medical image; combining the first segmented medical image and the second segmented medical image to obtain a segmented medical image, the segmented medical image comprising segmented normal tissue based on the first segmented medical image and segmented abnormal tissue based on the second segmented medical image.
Embodiment 18B: An apparatus for reviewing a medical image, the apparatus comprising: one or more processors; and memory accessible by the one or more processors, the memory storing instructions that when executed by the one or more processors, cause the apparatus to perform a method comprising: accessing from memory a medical image of a subject, the medical image comprising voxels; generating, using a first trained machine learning model and the medical image, a first segmented medical image, the first trained machine learning model trained to generate a medical image segmenting normal tissue in a medical image; generating, using a second trained machine learning model and the medical image, a second segmented medical image, the second trained machine learning model trained to generate a medical image segmenting abnormal tissue in a medical image; combining the first segmented medical image and the second segmented medical image to obtain a segmented medical image, the segmented medical image comprising segmented normal tissue based on the first segmented medical image and segmented abnormal tissue based on the second segmented medical image.
Embodiment 19: A computer-implemented method for reviewing a medical image, the method comprising: accessing from memory a medical image of a subject, the medical image comprising voxels; generating, using a first trained machine learning model and the medical image, a first segmented medical image, the first trained machine learning model trained to generate a medical image segmenting normal tissue in a medical image, the first segmented medical image comprising segmentation label data identifying voxels of the first segmented medical image as segmented normal tissue or non-segmented tissue; generating, using a second trained machine learning model and the medical image, a second segmented medical image, the second trained machine learning model trained to generate a medical image segmenting abnormal tissue in a medical image, the second segmented medical image comprising segmentation label data identifying voxels of the second segmented medical image as segmented abnormal tissue or non-segmented tissue; combining the first segmented medical image and the second segmented medical image to obtain a segmented medical image, the segmented medical image comprising segmented normal tissue based on the first segmented medical image and segmented abnormal tissue based on the second segmented medical image, the segmented medical image comprising segmentation label data based on the segmentation label data of the first segmented medical image and the segmentation label data of the second segmented medical image.
Embodiment 19A: A non-transitory processor readable medium containing a set of instructions thereon for reviewing a medical image, wherein when executed by a processor, the instructions cause the processor to perform a method comprising: accessing from memory a medical image of a subject, the medical image comprising voxels; generating, using a first trained machine learning model and the medical image, a first segmented medical image, the first trained machine learning model trained to generate a medical image segmenting normal tissue in a medical image, the first segmented medical image comprising segmentation label data identifying voxels of the first segmented medical image as segmented normal tissue or non-segmented tissue; generating, using a second trained machine learning model and the medical image, a second segmented medical image, the second trained machine learning model trained to generate a medical image segmenting abnormal tissue in a medical image, the second segmented medical image comprising segmentation label data identifying voxels of the second segmented medical image as segmented abnormal tissue or non-segmented tissue; combining the first segmented medical image and the second segmented medical image to obtain a segmented medical image, the segmented medical image comprising segmented normal tissue based on the first segmented medical image and segmented abnormal tissue based on the second segmented medical image, the segmented medical image comprising segmentation label data based on the segmentation label data of the first segmented medical image and the segmentation label data of the second segmented medical image.
Embodiment 19B: An apparatus for reviewing a medical image, the apparatus comprising: one or more processors; and memory accessible by the one or more processors, the memory storing instructions that when executed by the one or more processors, cause the apparatus to perform a method comprising: accessing from memory a medical image of a subject, the medical image comprising voxels; generating, using a first trained machine learning model and the medical image, a first segmented medical image, the first trained machine learning model trained to generate a medical image segmenting normal tissue in a medical image, the first segmented medical image comprising segmentation label data identifying voxels of the first segmented medical image as segmented normal tissue or non-segmented tissue; generating, using a second trained machine learning model and the medical image, a second segmented medical image, the second trained machine learning model trained to generate a medical image segmenting abnormal tissue in a medical image, the second segmented medical image comprising segmentation label data identifying voxels of the second segmented medical image as segmented abnormal tissue or non-segmented tissue; combining the first segmented medical image and the second segmented medical image to obtain a segmented medical image, the segmented medical image comprising segmented normal tissue based on the first segmented medical image and segmented abnormal tissue based on the second segmented medical image, the segmented medical image comprising segmentation label data based on the segmentation label data of the first segmented medical image and the segmentation label data of the second segmented medical image.
Embodiment 20: A computer-implemented method for reviewing a medical image, the method comprising: accessing from memory a medical image of a subject, the medical image comprising voxels; generating, using a first trained machine learning model and the medical image, a first segmented medical image, the first trained machine learning model trained to generate a medical image segmenting normal tissue in a medical image, the first segmented medical image comprising uncertainty measures of segmentation label data; generating, using a second trained machine learning model and the medical image, a second segmented medical image, the second trained machine learning model trained to generate a medical image segmenting abnormal tissue in a medical image, the second segmented medical image comprising uncertainty measures of segmentation label data; combining the first segmented medical image and the second segmented medical image to obtain a segmented medical image based on the uncertainty measures of the first segmented medical image and the uncertainty measures of the second segmented medical image, the segmented medical image comprising segmented normal tissue based on the first segmented medical image and segmented abnormal tissue based on the second segmented medical image.
Embodiment 20A: A non-transitory processor readable medium containing a set of instructions thereon for reviewing a medical image, wherein when executed by a processor, the instructions cause the processor to perform a method comprising: accessing from memory a medical image of a subject, the medical image comprising voxels; generating, using a first trained machine learning model and the medical image, a first segmented medical image, the first trained machine learning model trained to generate a medical image segmenting normal tissue in a medical image, the first segmented medical image comprising uncertainty measures of segmentation label data; generating, using a second trained machine learning model and the medical image, a second segmented medical image, the second trained machine learning model trained to generate a medical image segmenting abnormal tissue in a medical image, the second segmented medical image comprising uncertainty measures of segmentation label data; combining the first segmented medical image and the second segmented medical image to obtain a segmented medical image based on the uncertainty measures of the first segmented medical image and the uncertainty measures of the second segmented medical image, the segmented medical image comprising segmented normal tissue based on the first segmented medical image and segmented abnormal tissue based on the second segmented medical image.
Embodiment 20B: An apparatus for reviewing a medical image, the apparatus comprising: one or more processors; and memory accessible by the one or more processors, the memory storing instructions that when executed by the one or more processors, cause the apparatus to perform a method comprising: accessing from memory a medical image of a subject, the medical image comprising voxels; generating, using a first trained machine learning model and the medical image, a first segmented medical image, the first trained machine learning model trained to generate a medical image segmenting normal tissue in a medical image, the first segmented medical image comprising uncertainty measures of segmentation label data; generating, using a second trained machine learning model and the medical image, a second segmented medical image, the second trained machine learning model trained to generate a medical image segmenting abnormal tissue in a medical image, the second segmented medical image comprising uncertainty measures of segmentation label data; combining the first segmented medical image and the second segmented medical image to obtain a segmented medical image based on the uncertainty measures of the first segmented medical image and the uncertainty measures of the second segmented medical image, the segmented medical image comprising segmented normal tissue based on the first segmented medical image and segmented abnormal tissue based on the second segmented medical image.
Embodiment 21: A method, machine, manufacture, and/or system substantially as shown and described.

Embodiments illustrated under any heading or in any portion of the disclosure may be combined with embodiments illustrated under the same or any other heading or other portion of the disclosure unless otherwise indicated herein or otherwise clearly contradicted by context. For example, and without limitation, embodiments described in dependent claim format for a given embodiment (e.g., the given embodiment described in independent claim format) may be combined with other embodiments (described in independent claim format or dependent claim format).

Numerous modifications, alterations, and changes to the described embodiments are possible without departing from the scope of the present invention defined in the claims. It is intended that the present invention not be limited to the described embodiments, but that it has the full scope defined by the language of the following claims, and equivalents thereof.

What is claimed is:

1. A computer-implemented method for reviewing a medical image, the method comprising:
accessing from memory a medical image of a subject, the medical image comprising voxels;
generating, using a first trained machine learning model and the medical image, a first segmented medical image, the first trained machine learning model trained to generate a medical image segmenting normal tissue in a medical image;
generating, using a second trained machine learning model and the medical image, a second segmented medical image, the second trained machine learning model trained to generate a medical image segmenting abnormal tissue in a medical image;
combining the first segmented medical image and the second segmented medical image to obtain a segmented medical image, the segmented medical image comprising segmented normal tissue based on the first segmented medical image and segmented abnormal tissue based on the second segmented medical image.

2. The method of claim 1, wherein the first segmented medical image comprises voxels having segmentation label data, the segmentation label data comprising one of a segmented normal tissue label or a non-segmented tissue label, and
wherein the second segmented medical image comprises voxels having segmentation label data, the segmentation label data comprising one of a segmented abnormal tissue label or a non-segmented tissue label.

3. The method of claim 2, wherein the segmentation label data of the first segmented medical image further comprises a label uncertainty measure, and
wherein the segmentation label data of the second segmented medical image further comprises a label uncertainty measure.

4. The method of claim 1, wherein combining the first segmented medical image and the second segmented medical image to obtain the segmented medical image comprises prioritizing the second segmented medical image over the first segmented medical image.

5. The method of claim 1, wherein combining the first segmented medical image and the second segmented medical image to obtain the segmented medical image comprises:
for a voxel of the medical image, assigning segmentation label data for the voxel of the medical image based on segmentation label data of a corresponding voxel of either the first segmented medical image or the second segmented medical image.

6. The method of claim 1, wherein combining the first segmented medical image and the second segmented medical image to obtain the segmented medical image comprises:
for a voxel of the segmented medical image,
if a corresponding voxel of the second segmented medical image has a segmented abnormal tissue label, assigning the segmented abnormal tissue label to the voxel of the segmented medical image, and
if the corresponding voxel of the second segmented medical image does not have the segmented abnormal tissue label, and if a corresponding voxel of the first segmented medical image has a segmented normal tissue label, assigning the segmented normal tissue label to the voxel of the segmented medical image.

7. The method of claim 1, wherein for a voxel of the segmented medical image:
if a corresponding voxel of the second segmented medical image has a segmented abnormal tissue label, the voxel of the segmented medical image has the segmented abnormal tissue label of the corresponding voxel of the second segmented medical image, and
if the corresponding voxel of the second segmented medical image does not have the segmented abnormal tissue label, and if a corresponding voxel of the first segmented medical image has a segmented normal tissue label, the voxel of the segmented medical image has the segmented normal tissue label of the corresponding voxel of the first segmented medical image.

8. The method of claim 7, wherein for a voxel of the segmented medical image:
if the corresponding voxel of the second segmented medical image does not have a segmented abnormal tissue label, and if the corresponding voxel of the first segmented medical image does not have a segmented normal tissue label, the voxel of the segmented medical image has a non-segmented tissue label.

9. The method of claim 1, wherein combining the first segmented medical image and the second segmented medical image to obtain the segmented medical image comprises:
for a voxel of the medical image, assigning segmentation label data for the voxel of the medical image based on an uncertainty measure of segmentation label data of the corresponding voxel of either the first segmented medical image or the second segmented medical image.

10. The method of claim 1, wherein combining the first segmented medical image and the second segmented medical image to obtain the segmented medical image comprises:
for a voxel of the segmented medical image,
if a corresponding voxel of the second segmented medical image has an uncertainty measure less than or equal to an abnormal voxel threshold, assigning a segmented abnormal tissue label to the voxel of the segmented medical image, and
if the corresponding voxel of the second segmented medical image has an uncertainty measure greater than an abnormal voxel threshold, and if a corresponding voxel of the first segmented medical image has an uncertainty measure less than or equal to a normal voxel threshold, assigning the segmented normal tissue label to the voxel of the segmented medical image.

11. The method of claim 10, wherein combining the first segmented medical image and the second segmented medical image to obtain the segmented medical image further comprises:
for a voxel of the segmented medical image,
if a corresponding voxel of the second segmented medical image has an uncertainty measure greater than an abnormal voxel threshold, and if a corresponding voxel of the first segmented medical image has an uncertainty measure greater than a normal voxel threshold, assigning a non-segmented tissue label to the voxel of the segmented medical image.

12. The method of claim 10, wherein combining the first segmented medical image and the second segmented medical image to obtain the segmented medical image further comprises:
for a voxel of the segmented medical image,
if a corresponding voxel of the second segmented medical image has an uncertainty measure greater than a too-uncertain threshold, and if a corresponding voxel of the first segmented medical image has an uncertainty measure greater than a too-uncertain threshold, assigning a non-segmented tissue label to the voxel of the segmented medical image.

13. The method of claim 10, wherein the first segmented medical image comprises voxels having segmentation label data, the segmentation label data comprising one of a segmented normal tissue label or a non-segmented tissue label, the segmentation label data further comprising a label uncertainty measure for each label,
wherein the second segmented medical image comprises voxels having segmentation label data, the segmentation label data comprising one of a segmented abnormal tissue label or a non-segmented tissue label, the segmentation label data further comprising a label uncertainty measure for each label.

14. The method of claim 10, wherein combining the first segmented medical image and the second segmented medical image to obtain the segmented medical image further comprises:
receiving a user selection of the abnormal voxel threshold;
assigning the segmented abnormal tissue label to the voxel of the segmented medical image based on the user-selected abnormal voxel threshold;
receiving a user selection of the normal voxel threshold; and
assigning the segmented normal tissue label to the voxel of the segmented medical image based on the user-selected normal voxel threshold.

15. The method of claim 1, wherein the first trained machine learning model comprises a transformer neural network or a convolutional neural network, wherein the second trained machine learning model comprises a transformer neural network or a convolutional neural network.

16. The method of claim 1, wherein the first trained machine learning model is trained to generate a medical image segmenting normal tissue in a medical image and is not trained to generate a medical image segmenting abnormal tissue in a medical image,
wherein the second trained machine learning model is trained to generate a medical image segmenting abnormal tissue in a medical image and is not trained to generate a medical image segmenting normal tissue in a medical image.

17. The method of claim 1, wherein the normal tissue comprises at least one organ, and the abnormal tissue comprises at least one of a tumor, an improperly functioning organ, or a resection area.

18. The method of claim 1, further comprising:
defining a region of interest (ROI) in the medical image for application of tumor treating fields to the subject;
creating a three-dimensional model of the subject based on the segmented medical image, the three-dimensional model of the subject including the region of interest;
generating a plurality of transducer layouts for application of tumor treating fields to the subject based on the three-dimensional model of the subject;
selecting at least two of the transducer layouts as recommended transducer layouts;
presenting the recommended transducer layouts;
receiving a user selection of at least one recommended transducer layout; and
providing a report for the at least one selected recommended transducer layout.

19. A computer-implemented method for reviewing a medical image, the method comprising:
accessing from memory a medical image of a subject, the medical image comprising voxels;
generating, using a first trained machine learning model and the medical image, a first segmented medical image, the first trained machine learning model trained to generate a medical image segmenting normal tissue in a medical image, the first segmented medical image comprising segmentation label data identifying voxels of the first segmented medical image as segmented normal tissue or non-segmented tissue;
generating, using a second trained machine learning model and the medical image, a second segmented medical image, the second trained machine learning model trained to generate a medical image segmenting abnormal tissue in a medical image, the second segmented medical image comprising segmentation label data identifying voxels of the second segmented medical image as segmented abnormal tissue or non-segmented tissue;
combining the first segmented medical image and the second segmented medical image to obtain a segmented medical image, the segmented medical image comprising segmented normal tissue based on the first segmented medical image and segmented abnormal tissue based on the second segmented medical image, the segmented medical image comprising segmentation label data based on the segmentation label data of the first segmented medical image and the segmentation label data of the second segmented medical image.

20. A computer-implemented method for reviewing a medical image, the method comprising:
accessing from memory a medical image of a subject, the medical image comprising voxels;
generating, using a first trained machine learning model and the medical image, a first segmented medical image, the first trained machine learning model trained to generate a medical image segmenting normal tissue in a medical image, the first segmented medical image comprising uncertainty measures of segmentation label data;
generating, using a second trained machine learning model and the medical image, a second segmented medical image, the second trained machine learning model trained to generate a medical image segmenting abnormal tissue in a medical image, the second segmented medical image comprising uncertainty measures of segmentation label data;
combining the first segmented medical image and the second segmented medical image to obtain a segmented medical image based on the uncertainty measures of the first segmented medical image and the uncertainty measures of the second segmented medical image, the segmented medical image comprising segmented normal tissue based on the first segmented medical image and segmented abnormal tissue based on the second segmented medical image.
